(19) United States
(12) Patent Application Publication
(10) Pub. No.: US 2025/0136945 A1
(43) Pub. Date: May 1, 2025

(54) TISSUE CONTAINING MESENCHYMAL STEM CELLS AND APPARATUS AND METHOD FOR PROMOTING THE ANTI-INFLAMMATORY, ANTINOCICEPTIVE PROPERTIES AND FOR IMPROVING THE REPAIRING CAPABILITY OF A TISSUE CONTAINING MESENCHYMAL STEM CELLS

(71) Applicant: SURGERE S.R.L., Trento (IT)

(72) Inventor: Alfredo GORIO, Milano (IT)

(21) Appl. No.: 18/835,410

(22) PCT Filed: Feb. 3, 2022
(86) PCT No.: PCT/IB2022/050941

§ 371 Date: Aug. 2, 2024

(51) Int. Cl. C12N 5/0775; C12M 1/12; C12M 1/24; C12M 1/42; C12M 3/00

(52) U.S. Cl. CPC C12N 5/0667; C12M 23/06; C12M 23/08; C12M 23/42; C12M 35/04

(57) ABSTRACT

Provided is an apparatus for promoting anti-inflammatory properties and the repairing capability of a tissue containing mesenchymal stem cells. The apparatus has a containment and support device for washed lipoaspirated adipose tissue containing mesenchymal stem cells. The containment and support device has an external containment body which forms an access mouth closable by a closure lid, a transfer body being accommodatable within the external containment body and forming at least one locking seat for a respective syringe-like body which accommodates the washed lipoaspirated adipose tissue. A mechanical activation device has an orbital shaker which forms means for accommodating the containment and support device.

The present invention relates to an apparatus and a method for promoting the anti-inflammatory, antinociceptive properties and for improving the repairing capability of a tissue containing mesenchymal stem cells.

It is known to prepare tissues that can repair, replace or restore biological structures and/or functions that have been lost due to congenital defects, aging, disease or destruction thereof (Haseltine, 2003; Gutmann et al., 2005, Greenwood et al., 2006).

Mesenchymal stem cells (MSCs) are multipotent cells capable of self-renewal and with the capability to differentiate themselves, in vitro, into cells of mesenchymal origin, including osteoblasts, adipocytes and chondrocytes, and give rise to tissues such as bone, fat, cartilage and muscle, in vivo (Pettinger et al., 1999). Moreover, recent studies have shown that the tissue regeneration potential can be improved by using mesenchymal stem cells derived from human adipose tissue (so-called hADSCs) (Gimle et al., 2011).

Moreover, it has been found that the adipose tissue of obese people, with respect to slimmer individuals, exhibits a greater expression of proinflammatory proteins, including TNF-α, interleukin 6 (IL-6), monocyte chemotactic protein 1, growth factor β1, procoagulant proteins, such as type 1 plasminogen activator inhibitor, tissue factor, and factor VII.

Furthermore, the number of macrophages increases in the adipose tissue of obese individuals, where they apparently function to eliminate moribund adipocytes, the number of which increases exponentially with obesity.

Macrophages are probably the cause of most of the production of cytokines in the adipose tissue of obese persons. Resident macrophages in adipose tissue are in fact responsible for the production of almost all TNF-α adipose tissue, release large quantities of IL-6 and produce an inducible isoform of nitric oxide synthase. Att: esistono entrambe le definizioni e nel testo non ci sono altri punti per cui poter confrontare e verificare. Senza verificare alla fonte, non cambierei.

In particular, it has been observed that the expression of inflammation-specific genes by macrophages was increased in the adipose tissue of obese mice. This is preceded by an intense increase in the production of insulin. Moreover, when rosiglitazone, an insulin-sensitizing drug, was administered to those mice the expression of the genes cited above and others decreased. Therefore, the appropriate sequence of appearance of inflammatory molecules prior to the development of insulin resistance, as well as their known capability to promote both insulin resistance and other complications of obesity, strongly suggests that inflammation of adipose tissue has an important role in the development of obesity.

It is noted, moreover, that the application of a mechanical force for a few minutes abolishes the inflammatory properties of fats and favors the expression of anti-inflammatory proteins, such as TNF-stimulated gene-6 (TSG-6).

This anti-inflammatory cytokine has demonstrated protective effects when administered in experimental models of disease by means of the reduction of infiltrating neutrophils and the neutralization of CXCL8. This can render the use of adipose tissue in human clinical transplantation procedures much more reliable and capable of contributing to a positive result.

Indeed, autologous transplantation of adipose tissue is an established therapeutic procedure used to repair various types of tissue damage. In order to minimize discrepancies and inconsistencies and allow the comparison of the data generated in different laboratories, the International Society of Cellular Therapy (ISCT) has provided guidelines for the definition of mesenchymal stem cells (MSC) based on their (a) plastic adhesion property; (b) immunophenotype; and (c) multipotent differentiation potential.

Clinical and preclinical studies show that autologous MSCs survive after transplantation, exhibit pluripotent differentiation and have antiapoptotic, anti-inflammatory and radiogenic effects.

Furthermore, there is evidence that mesenchymal stem cells (hADSC) derived from mechanically activated human adipose tissue grow well in culture, since they maintain their karyotype, their differentiation is normal and most of all these cells exhibit much higher anti-inflammatory properties than those derived from adipose tissue not stimulated mechanically.

Samples of lipoaspirated human adipose tissue have been obtained from elective liposuction procedures performed under local anesthesia with lidocaine (AstraZeneca, London, UK). This procedure provides for an infiltration step, in which a solution of saline solution and epinephrine (2 μg/mL) (Key Customer Solutions SAS, Basiglio, Milan, Italy) is infused in the adipose compartment in order to minimize blood loss and reduce the infiltration of peripheral blood cells. The product obtained with this procedure was termed lipoaspirated adipose tissue (lipoaspirate).

A method is known, from European patent application EP3397754 in the name of Prof. Alfredo Gorio, for promoting anti-inflammatory properties and for improving the repair ability of a tissue containing mesenchymal stem cells (MSCs) which provides for a step of mechanical activation performed by applying an orbital shaking force to adipose tissue lipoaspirated or removed from the body.

As clearly pointed out in the cited patent application, which is referenced herein as regards both the background art and the outcomes of the experimental procedures, transplantation of tissue and/or mesenchymal stem cells derived from adipose tissue and activated mechanically by orbital shaking can promote rapid restoration of motor function of animals that have lost or reduced this mobility.

In particular, as observed, activated adipose tissue is very effective in promoting functional recovery after medullary lesion, thanks to its anti-inflammatory properties and its ability to penetrate the lesion and promote nerve regeneration.

The aim of the present invention is to provide an apparatus and a method for promoting anti-inflammatory properties and for improving the repairing capability of a tissue containing mesenchymal stem cells that are capable of improving the background art in one or more of the aspects indicated above.

Within this aim, an object of the invention is to provide an apparatus and a method for promoting anti-inflammatory properties and for improving the repairing capability of a tissue containing mesenchymal stem cells that are extremely safe and practical.

Another object of the invention is to devise an apparatus and a method for promoting anti-inflammatory properties and for improving the repairing capability of a tissue containing mesenchymal stem cells that are capable of avoiding possible contaminations of the tissue.

Not least object of the invention is to provide an apparatus and a method for promoting anti-inflammatory properties and for improving the repairing capability of a tissue containing mesenchymal stem cells that are highly reliable, relatively easy to provide and at competitive costs.

This aim, as well as these and other objects which will become better apparent hereinafter, are achieved by an apparatus and a method for promoting anti-inflammatory properties and for improving the repairing capability of a tissue containing mesenchymal stem cells according to the independent claims, optionally having one or more of the characteristics of the dependent claims.

Further characteristics and advantages of the invention will become better apparent from the description of a preferred but not exclusive embodiment of the apparatus and a method for promoting anti-inflammatory properties and for improving the repairing capability of a tissue containing mesenchymal stem cells according to the invention, illustrated by way of non-limiting example in the accompanying drawings, wherein:

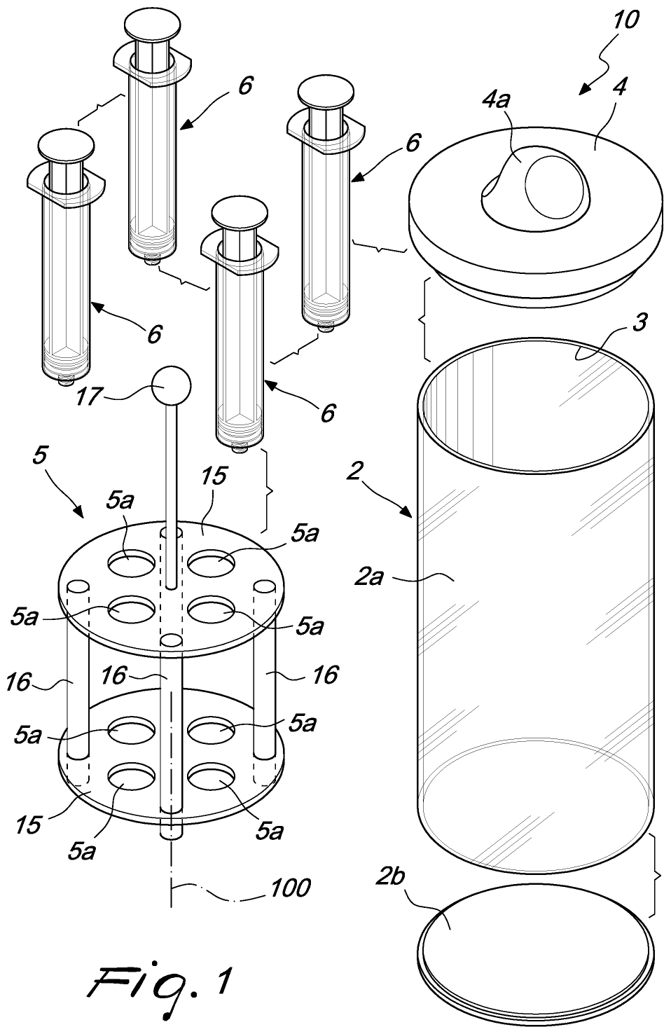

FIG. 1 is an exploded perspective view of the apparatus according to the invention;

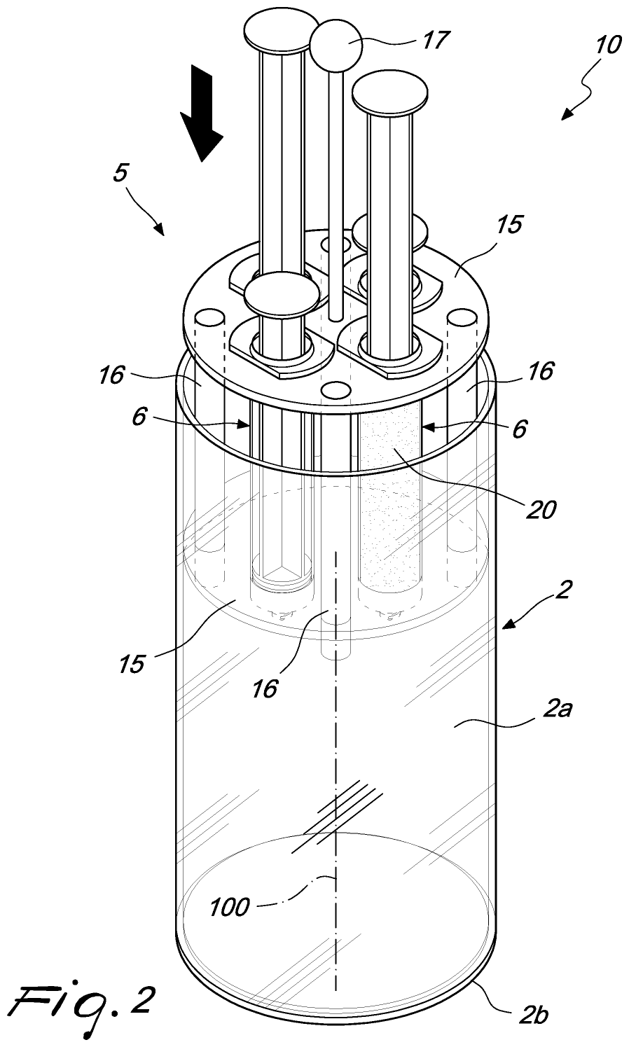

FIG. 2 is again a perspective view of the insertion of the transfer body in the external containment body 2;

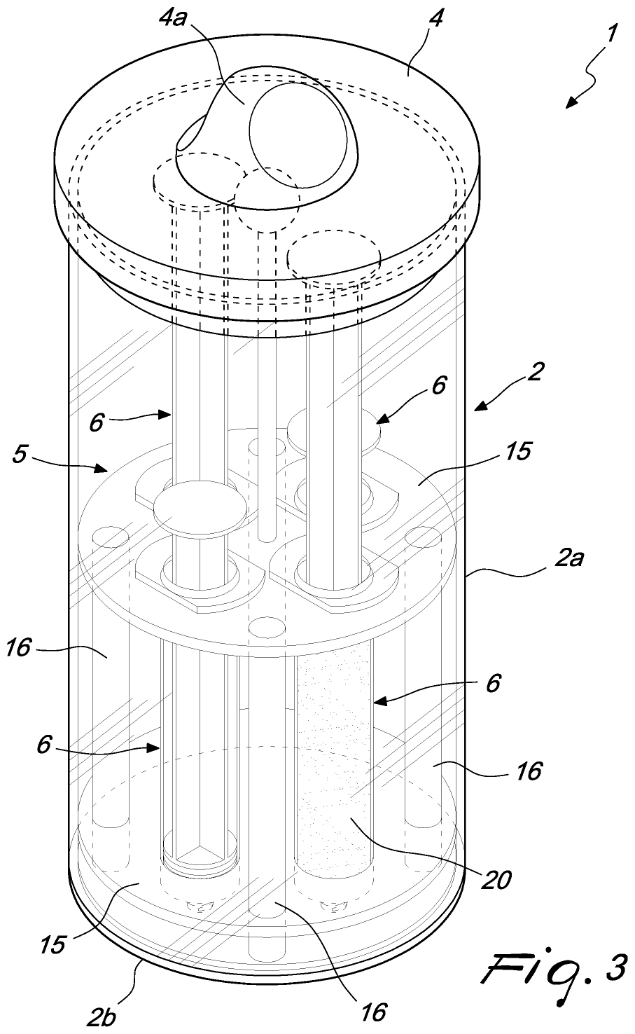

FIG. 3 is a perspective view, in phantom lines, of the containment and support device.

With reference to the figures, the apparatus for promoting anti-inflammatory properties and for improving the repairing capability of a tissue containing mesenchymal stem cells according to the invention comprises a containment and support device 10 for washed lipoaspirated adipose tissue containing mesenchymal stem cells.

The containment and support device 10 comprises an external containment body 2 which forms an access mouth 3 which can be closed by means of a closure lid 4.

The external containment body 2 has, on the opposite side with respect to the access mouth 3, a closure wall 2b.

Conveniently, the closure lid 4 è configured to ensure a hermetic closure of the access mouth 3.

Advantageously, the closure lid 4 is further provided with a grip portion 4a to facilitate its opening and closing.

Inside the external containment body 2 it is possible to accommodate a transfer body 5, which forms at least one locking seat 5a for a respective syringe-like body 6 which accommodates the washed lipoaspirated adipose tissue.

Conveniently, the transfer body 5 comprises at least two plates 15 which are mutually connected by means of posts 16.

The plates 15 are furthermore associated with a grip element 17 to facilitate the insertion and extraction of the transfer body 5 in and from the external containment body 2.

The apparatus further comprises a mechanical activation device which comprises an orbital shaker, not shown, and forms means for accommodating the containment and support device 10.

Advantageously, the external containment body 2 has a substantially cylindrical or frustum-like extension which extends along a longitudinal axis of extension 100.

The transfer body 5 forms at least two locking seats 5a, which are mutually uniformly angularly spaced, during use, along the axis of extension 100 of the external containment body 2.

According to one practical embodiment, shown in the figures, the transfer body 5 forms four locking seats 5a.

The locking seats 5a can be constituted by respective holes 25 provided in the two plates 15 and aligned with respect to the longitudinal axis of extension 100.

Preferably, when the transfer body 5 is accommodated inside the external containment body 2, the grip element 17 extends at the axis of extension 100 of the external containment body 2.

Conveniently, the syringe-like body 6 is configured to be connected stably to a so-called “Coleman type” fat separation kit.

In greater detail, the syringe-like body 6 comprises a male “Luer Lok” connection.

According to a further aspect, the apparatus according to the invention comprises an accommodation device (also known as secondary container) for the external containment body 2.

The accommodation device (secondary container) can be associated detachably with the mechanical activation device comprising an orbital shaker.

According to a further aspect, the present invention relates to a method for promoting anti-inflammatory properties and improving the repairing capability of a tissue containing mesenchymal stem cells, characterized in that it comprises:
a step of accommodation of washed lipoaspirated adipose tissue containing mesenchymal stem cells in at least one syringe-like body 6;
a step of positioning of said at least one syringe-like body 6 at a respective locking seat 5a formed on a transfer body 5;
a step of insertion of the transfer body 5 in an external containment body 2 through an access mouth 3;
a step of closure, by means of a closure lid 4, of the external containment body 2;
a step of positioning of the external containment body 2 in a mechanical activation device comprising an orbital shaker;
a step of application of an orbital shaking force to the adipose tissue.

Preferably, the method according to the invention provides, after the closure step, a step of accommodation of the external containment body 2 in an accommodation device.

The positioning step comprises a step of detachable association of the accommodation device with the orbital shaker.

Conveniently, the orbital or eccentric shaking force acting on the adipose tissue has an intensity comprised between 10×g and 180×g and advantageously 40×g and 120×g.

Advantageously, the orbital shaking force acting on the adipose tissue is applied for a time comprised between 1 minute and 35 minutes, and preferably between 3 minutes and 25 minutes and more preferably between 3 minutes and 10 minutes.

According to a further aspect, the present invention relates to an adipose tissue lipoaspirated or removed from the body containing mesenchymal stem cells obtained or obtainable with a method as described above.

The invention furthermore relates to a tissue for use in the treatment of human spinal traumas.

It has been found experimentally that the tissue for use in the treatment of human spinal traumas is even more effective if said treatment further comprises combination with erythropoietin.

Specifically, said treatment further comprises the combination with 35,000 units of erythropoietin (hereinafter EPO) by slow infusion in the first two postoperative days.

In support of what has been highlighted above, the following tests were performed.

The left sciatic nerve of 48 rats was subjected to resection at the mid-thigh level.

One drop (250 microliters) of activated fat, activated for example by using the activation process described above, was placed in the lesion site; the two stumps of sciatic nerve were then sutured together in 24 rats.

The activated fat was also placed all around the nerve lesion for a length of 5 mm. The animals, subjected to treatment with activated fat, were also treated with 500 units/kg of erythropoietin administered intravenously right after the surgical procedure and a second time the next day, 24 hours after the lesion. The sciatic nerve of the other 24 rats received the same kind of lesion and was treated with saline.

Recovery of the innervation of the extensor digitorum longus (EDL) muscle was monitored at 3, 20, 28, 36, 44 and 50 days after the lesion of the nerve. In order to evaluate recovery, the indirect isometric tension of the EDL muscle following sciatic nerve stimulation was determined.

The average maximum contraction force of the EDL was 16.2 newtons (N), after the injury it decreased to 0 N. Contraction recovery began when reinnervation occurred. In the lesioned control rats, reinnervation began around day 21 and reached values close to the normal value in isometric tension on day 50. Treatment with activated fat of the lesioned nerve produced a faster and stronger reinnervation right from the first steps up to day 20. Muscle strength was much greater and very significantly higher in animals treated with activated fat during the reinnervation process from day 20 to day 44.

| Saline (N) | Treated (N) | Days from Injury |
|---|---|---|
| 16.2 | 16.2 | 0 |
| 0.0 | 0.0 | 3 |
| 0.0 | 1.4 | 20 |
| 3.4 | 9.2 | 28 |
| 6.5 | 13.5 | 36 |
| 10.3 | 16.5 | 44 |
| 14.4 | 16.9 | 50 |

In conclusion, the combination of activated fat on the lesioned nerves and the intravenous injection of EPO stimulated nerve regeneration and led to a more efficient recovery of leg muscle reinnervation.

This effect was also observed when such a treatment was applied to injured nerve roots in humans. A patient with a lesion at the root of the right sciatic nerve showed lack of ability to control the toes of the right foot and lacked sensory perceptions in the same foot. After 6 months of treatment with activated fat on the nerve roots and 35,000 units of EPO administered intravenously, full sensitivity of the foot and correct mobility of the toes of the right foot was achieved. This demonstrates that the treatment is effective in humans as well.

This is reinforced by the stimulated recovery of the functions of the urinary bladder and of the anus in patients with spinal cord lesions. The lesion site of 10 patients was treated with activated fat sourced from the same patient and EPO was supplied intravenously to 35,000 units by slow infusion. The treatment was repeated 3 more times: the following day and 35 and 60 days after spinal surgery. Restoration of bladder and anal function is mediated by the regeneration of the 5-HT axons that regrow from the lesion site to the sacral cord. Upon restoration of serotoninergic innervation of the sacral cord there is a gradual recovery of bladder and anal function. Six months after surgery on the spinal column of the patient and treatment with activated fat and EPO we observed recovery of the bladder and of defecation in these patients. Instrumental examination showed recovery of their function in all the patients. Perception of bladder fullness and of the degree of urgency was directly correlated to bladder volume. It is interesting to note that at the same level of bladder fullness and at the same urgency score at higher volumes of the patient, fullness of the bladder of 10/10 patients was perceived at 500 ml in hospital control. Moreover, in 5 patients out of ten motor activity passed from an ASIA A motor scale value to ASIA B in approximately three months, whereas in controls it remained ASIA A. Patients treated with activated fat and EPO were able to walk with the aid of a walker which allowed them to stand up with their own strength and to move their legs forward by virtue of the reinnervation of part of the muscles above the knee, as revealed by neurological control.

In conclusion: the conditions caused by lesions of the peripheral nerves and lesions of the spinal cord were improved considerably by the local application, in the lesion site, of activated fat and with the administration of EPO intravenously (i.v.). This effect occurred both through anti-inflammatory actions and by promotion of neuroregeneration.

Activated fat can also be obtained by means of different processes, such as for example the one described in European patent application EP3397754.

The present invention also relates to a kit comprising:
a) an adipose tissue lipoaspirated or removed from the body containing mesenchymal stem cells, obtained with a method for promotion of anti-inflammatory properties and for improving the repairing capability comprising a step of mechanical activation performed by means of the application of an orbital and/or eccentric shaking force to said adipose tissue lipoaspirated or removed from the body;
b) erythropoietin.

The invention also relates to an adipose tissue lipoaspirated or removed from the body containing mesenchymal stem cells, obtained with a method for promoting anti-inflammatory properties and for improving the repairing capability, comprising a step of mechanical activation performed by means of the application of an orbital and/or eccentric shaking force to the adipose tissue lipoaspirated or removed from the body, for use in the treatment of spinal traumas, in combination with erythropoietin.

In particular, it has been found from tests conducted that the mechanically activated adipose tissue according to the present invention, applied at the injured cord, gradually penetrates, filling the lesion region and acting as a bridge between the two stumps and thus allowing nerve regeneration, as clearly highlighted and explained in the Gorio patent.

The tissue and/or the mesenchymal stem cells obtained with the method and the apparatus according to the present invention may furthermore be:
for use as medical remedy;
for use in the treatment of motor and coordination dysfunctions;
for use in the treatment of Parkinson's disease;
for use in the treatment of medullary lesions.

In practice it has been found that the invention achieves the intended aim and objects, providing an apparatus and a method for promoting anti-inflammatory and antinociceptive properties, and for improving the tissue repairing capability on the part of a tissue containing mesenchymal stem cells.

In particular, it has been found that the use of the apparatus and method for promoting anti-inflammatory properties and for improving the repairing capability of a tissue containing mesenchymal stem cells allows to obtain more consistent and repeatable results, allowing to obtain an activated adipose tissue with high anti-inflammatory properties and at the same time the suppression of the inflammatory agents produced by fats.

These repeatability and effectiveness aspects are amplified by virtue of the assurance of noncontamination.

It has been verified experimentally that the described tissue containing mesenchymal stem cells is capable of promoting recovery of the function of the bladder and of the lower limbs, even in humans, after paralysis due to lesion of the spinal cord.

The invention thus conceived is susceptible of numerous modifications and variations, all of which are within the scope of the accompanying claims; all the details may furthermore be replaced with other technically equivalent elements.

In practice, the materials used, so long as they are compatible with the specific use, as well as the contingent shapes and dimensions, may be any according to the requirements and the state of the art.

Where technical features mentioned in any claim are followed by reference signs, those reference signs have been included for the sole purpose of increasing the intelligibility of the claims and accordingly such reference signs do not have any limiting effect on the interpretation of each element identified by way of example by such reference signs.

1-4. (canceled)

5. An apparatus for promoting anti-inflammatory, antinociceptive properties and the repairing capability of a tissue containing mesenchymal stem cells, wherein the apparatus comprises a containment and support device for washed lipoaspirated adipose tissue containing mesenchymal stem cells, said containment and support device comprising an external containment body which forms an access mouth closable by a closure lid, a transfer body being accommodatable within said external containment body and forming at least one locking seat for a respective syringe-like body which accommodates said washed lipoaspirated adipose tissue, and wherein the apparatus further comprises a mechanical activation device comprising an orbital shaker which forms means for accommodating said containment and support device.

6. The apparatus of claim 5, wherein said external containment body has a substantially cylindrical or frustum-like extension which is extended around a longitudinal axis of extension, said transfer body forming at least two locking seats which are angularly mutually uniformly spaced, during use, around the longitudinal axis of extension of said external containment body.

7. The apparatus of claim 5, wherein said syringe-like body is configured to be connected stably to a so-called “Coleman type” separation kit.

8. The apparatus of claim 5, wherein said syringe-like body comprises a male Luer-Lok connection.

9. The apparatus of claim 5, further comprising an accommodation device for said external containment body, said accommodation device being detachably associable with said mechanical activation device comprising the orbital shaker.

10. A method for promoting anti-inflammatory properties and the repairing capability of a tissue containing mesenchymal stem cells, the method comprising:
a step of accommodating washed lipoaspirated adipose tissue containing mesenchymal stem cells within at least one syringe-like body;
a step of positioning said at least one syringe-like body at a respective locking seat formed on a transfer body;
a step of inserting said transfer body within an external containment body through an access mouth;
a step of closing, by a closure lid, said external containment body;
a step of positioning said external containment body in a mechanical activation device comprising an orbital shaker; and
a step of applying an orbital shaking force to said washed lipoaspirated adipose tissue.

11. The method of claim 10, further comprising, after said closing step, a step of accommodating said external containment body within an accommodation device, said positioning step comprising a step of detachably associating said accommodation device with said orbital shaker.

12. The method of claim 10, wherein the orbital shaking force has an intensity comprised between 40×g and 120×g.

13. The method of claim 10, wherein the orbital shaking force is applied for a time comprised between 3 minutes and 10 minutes.
